(19) United States
(12) Patent Application Publication
(10) Pub. No.: US 2026/0092846 A1
(43) Pub. Date: Apr. 2, 2026

(54) FAST PENETRATION PROBE FOR CHARACTERIZING THE RHEOLOGICAL PROPERTIES OF CONCRETE

(71) Applicants: Nicolas Libre, Rolla, MO (US); Ali Fasihi, Rolla, MO (US)

(72) Inventors: Nicolas Libre, Rolla, MO (US); Ali Fasihi, Rolla, MO (US)

(73) Assignee: The Curators of the University of Missouri, Columbia, MO (US)

(21) Appl. No.: 19/343,329

(22) Filed: Sep. 29, 2025

Related U.S. Application Data
(60) Provisional application No. 63/700,460, filed on Sep. 27, 2024.

(51) Int. Cl. G01N 3/42; G01N 3/06; G01N 33/38

(52) U.S. Cl. CPC G01N 3/42; G01N 3/06; G01N 33/383

(57) ABSTRACT

A method of acquiring and analyzing properties of a fresh or partially set cement-based material includes penetrating the material with a probe driven by a controller-regulated actuator; measuring, with a force sensor, penetration forces applied to the probe; transmitting, from the sensor, force data to the controller; determining, by the controller, penetration depths based on the force data and/or actuator position data; recording, by the controller, the depths; associating, by the controller, the depths and the forces to form synchronized pairs; determining, by a processor, the yield force of the material based on change in relationship between the depth and the force within the pairs, and calculating, by the processor, a static yield stress of the material based at least in part on the yield force, a probe geometry factor, and an angle of internal friction of the material. A portable penetration device is also disclosed.

### CROSS-REFERENCE TO RELATED APPLICATION

### 1. Priority Application

The present application claims priority from U.S. Provisional Patent Application No. 63/700,460, filed Sep. 27, 2024, and entitled FAST PENETRATION PROBE FOR CHARACTERIZING THE RHEOLOGICAL PROPERTIES OF CONCRETE, the entire disclosure of which is hereby incorporated by reference herein.

### BACKGROUND OF THE INVENTION

### 1. Field of the Invention

The present invention relates generally to an apparatus and a testing and analysis method for characterizing the rheological properties of fresh cementitious (concrete or other cement-based) materials. More particularly, the present invention relates to a fast penetration probe and a testing and analysis method utilizing the fast penetration probe and data acquired therefrom, including the force-displacement response during controlled probe insertion.

### 2. Discussion of the Prior Art

Use of concrete or other cement-based materials in construction and other applications is well known. It is desirable for appropriate workability, printability, setting times, necessary post-setting structural characteristics, and so on that the material composition be correct. For instance, the composition should be neither too dry nor too watery, neither too smooth nor too densely populated by aggregate, etc.

One conventional approach to testing fresh properties of a cement-based material is known as a slump test. Although those skilled in the art may make necessary or preferred adjustments to the material based on such a test, such adjustments lack precision and scientific rigor.

Another conventional approach characterizes the static yield stress of a material using a rotational rheometer. In this approach, a sample is typically removed from the construction site or concrete mixer and tested in a controlled laboratory environment. Rigorous analysis may be made, but such analysis is time consuming and requires a non-portable laboratory setup. Furthermore, results are significantly time-delayed.

### SUMMARY

According to one aspect of the present invention, a method of acquiring and analyzing properties of a fresh or partially set cement-based material includes the steps of: (a) penetrating the material with a probe driven at a constant penetration speed by an actuator regulated by a controller; (b) for each of a plurality of sampling times, measuring, with a force sensor, a penetration force applied to the probe; (c) transmitting, from the force sensor, force data associated with the measured penetration forces to the controller; (d) for each of the plurality of sampling times, determining, by the controller, a probe penetration depth based on one or more of the following: the force data and actuator position data, wherein the actuator position data is received from the actuator or inferred by the controller; (e) recording, by the controller, the determined probe penetration depth; (f) for each of the plurality of sampling times, associating, by the controller, the probe penetration depth and the penetration force to form a synchronized pair; (g) determining, by a processor, a yield force of the material based on change in relationship between the penetration depth and the penetration force within the synchronized pairs; and (h) calculating a static yield stress of the material by the processor, based at least in part on the yield force, a probe geometry factor, and an angle of internal friction of the material.

According to another aspect of the present invention, a portable penetration device for in-situ testing and characterization of a cement-based material comprises a probe configured to penetrate the material, an actuator for controlling vertical displacement of the probe, a force sensor mounted relative to the probe and configured to measure forces exerted on the probe, a controller for synchronizing force and displacement data associated with the probe to form synchronized pairs, and a processor for calculating and outputting a yield stress based on the synchronized pairs.

This summary is provided to introduce a selection of concepts in a simplified form. These concepts are further described below in the detailed description of the preferred embodiments. This summary is not intended to identify key features or essential features of the claimed subject matter, nor is it intended to be used to limit the scope of the claimed subject matter.

Various other aspects and advantages of the present invention will be apparent from the following detailed description of the preferred embodiments and the accompanying drawing figures.

### BRIEF DESCRIPTION OF THE DRAWING FIGURES

Preferred embodiments of the present invention are described in detail below with reference to the attached drawing figures, wherein:

FIG. 1 is a perspective view of a portable penetration device in accordance with a first preferred embodiment of the present invention, along with a material sample for testing;

FIG. 2 is schematic diagram of the portable penetration device and material sample of FIG. 1, particularly illustrating the interconnectivity of the various electronics and computational components of the penetration device;

FIG. 3 is an enlarged perspective view of the force sensor and probe shaft of FIGS. 1 and 2, with a first alternative probe tip attached to the probe shaft;

FIG. 4 is a perspective view similar to FIG. 3, but with a second alternative probe tip attached to the probe shaft and with the probe including a sleeve;

FIG. 5 is a side view of a three-dimensional concrete printer and of a pair of penetration devices in accordance with a second preferred embodiment of the present invention, wherein the penetration devices are similar to the penetration device of FIGS. 1-4 but configured for mounting on the printer;

FIG. 6 is a partially sectioned perspective view of a portable penetration device in accordance with a third preferred embodiment of the present invention, in addition to a container for a material sample for testing;

FIG. 7 is a front view of the penetration device and container of FIG. 6; and

FIG. 8 is perspective view of a partially sectioned concrete slab and associated framing, in addition to a penetration device in accordance with a fourth preferred embodiment of the present invention, wherein the penetration device is similar to the penetration device of FIGS. 6 and 7 but with a modified frame to enable mounting on the framing.

The drawing figures do not limit the present invention to the specific embodiments disclosed and described herein. Furthermore, the drawings do not necessarily provide exact dimensions or tolerances for the illustrated structures or components, and in some instances may not be to scale with respect to the relationships between the components of the structures illustrated in the drawings.

### DETAILED DESCRIPTION

The present invention is susceptible of embodiment in many different forms. While the drawings illustrate, and the specification describes, certain preferred embodiments of the invention, it is to be understood that such disclosure is by way of example only. There is no intent to limit the principles of the present invention to the particular disclosed embodiments.

Furthermore, unless specified or made clear, the directional references made herein with regard to the present invention and/or associated components (for instance, top, bottom, upper, lower, inner, outer, and so on) are used solely for the sake of convenience and should be understood only in relation to each other. For instance, a component might in practice be oriented such that faces referred to as “top” and “bottom” are sideways, angled, inverted, and so on relative to the chosen frame of reference.

### First Embodiment: Rotationally Actuated Portable Penetration Device

### Overview

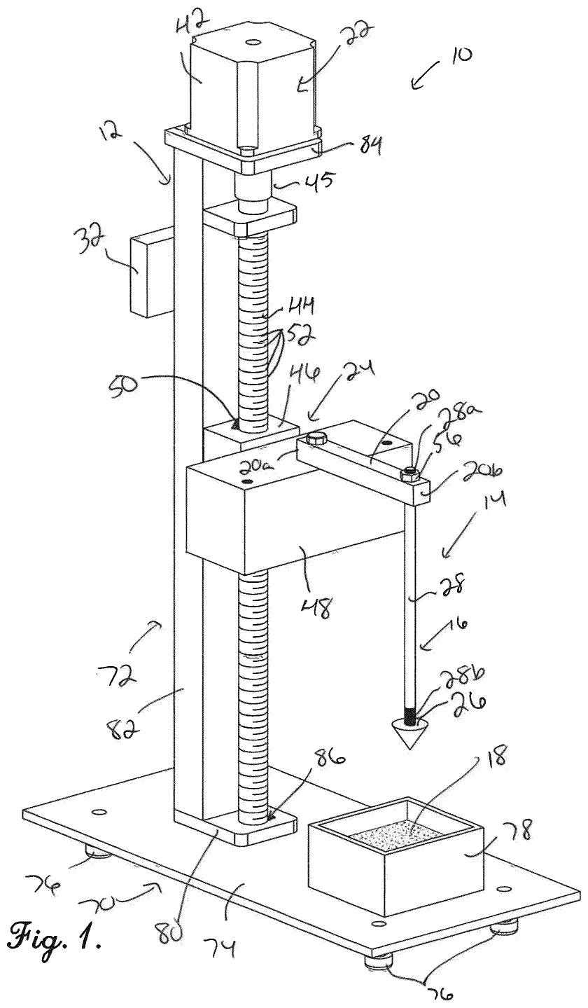

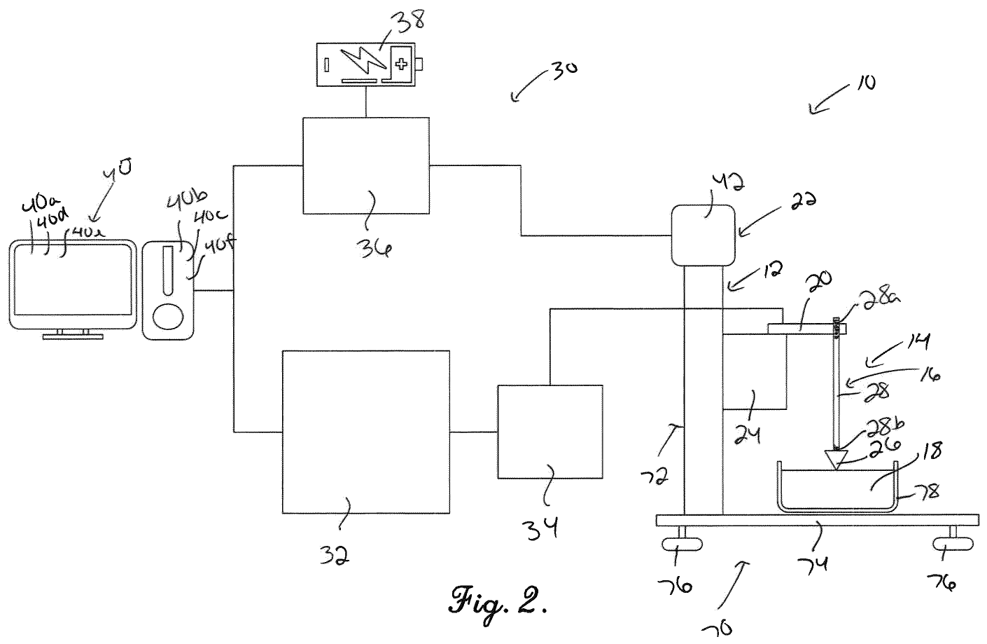

A first preferred embodiment of the present invention is shown in FIGS. 1 and 2. More particularly, a portable penetration device 10 is shown. The device 10 broadly includes a housing or frame 12 and a penetration system 14 supported by the frame 12.

The penetration system 14 broadly includes a probe 16 configured to contact or penetrate a test material 18, a force sensor 20 mounted relative to the probe 16, an actuator 22 configured to control the displacement of the probe 16, and an adapter 24 operably connecting the probe 16 to the actuator 22.

The probe 16 includes a probe tip 26 and a holder or probe shaft 28 to which the probe tip 26 is mounted. As will be described in greater detail below, the probe shaft 28 is operably interconnected to the force sensor 20, with the force sensor 20 being configured to measure forces exerted on the probe tip 26 during contact with and/or penetration of the material 18.

As shown schematically in FIG. 2, the penetration system 14 further preferably includes a plurality of electronic and computational components 30, including a controller or control board 32 (also shown in FIG. 1), a force sensor amplifier 34, a motor driver 36, a power supply 38, and a user system 40.

### Actuator

In the illustrated embodiment, the actuator 22 is configured to convert rotatory motion into precise linear motion. More particularly, the actuator 22 of the illustrated embodiment includes a motor 42, a threaded shaft or lead screw 44, and a coupler 45 operably interconnecting the motor 42 and the lead screw 44. The adapter 24 includes an adapter block 46 mounted to the actuator shaft 44 and an extension body 48 projecting from the adapter block 46. The adapter block 46 defines an opening 50 in which the actuator shaft 44 is received. The opening 50 is an internally threaded opening 50, with the threads thereof (not shown) corresponding to external threads 52 of the actuator shaft 44.

Rotation of the actuator shaft 44, as driven by the motor 42, results in linear shifting of the adapter 24 and, as will be discussed in greater detail below, of the probe 16, along the actuator shaft 44. Both upward and downward motion are facilitated.

In a preferred embodiment of the present invention, the motor 42 is a stepper motor. For instance, the motor 42 in a preferred embodiment is a National Electrical Manufacturers Association (NEMA) 23 stepper motor with an integrated gearbox to provide a holding torque up to four (4) Nm. Other motors, including non-stepper motors, fall within the scope of some aspects of the present invention, however.

The stepper motor driver 36 preferably receives low-voltage pulse commands from the controller 32 and delivers precisely timed high-current pulses to the stepper motor 42 to achieve accurate displacement control.

It is noted that the actuator stroke length is variable to accommodate testing to different material depths. Furthermore, the probe 16 progresses linearly at user-defined penetration speeds. For instance, in a preferred embodiment of the present invention, the penetration speed can be adjusted between one hundredth (0.01) mm/s and fifteen (15) mm/s. A penetration speed of between about five tenths (0.5) mm/s and about seven and five tenths (7.5) mm/s is preferred in most testing methodologies, however, with a penetration speed of about five tenths (0.5) mm/s being most preferred.

### Force Sensor

The force sensor 20 includes proximal and distal ends 20a and 20b. The force sensor 20 is mounted at its proximal end 20 to the extension body 48, which is in turn attached to the adapter block 46.

As will be discussed in greater detail below, the probe 16 is mounted to the distal end 20b of the force sensor 20.

As will also be discussed in greater detail below, the force sensor 20 senses forces exerted on the probe 16 during penetration of the test material 18, enabling accurate data collection.

In the illustrated embodiment, the force sensor 20 comprises a bending beam type load cell, although other force sensor types fall within the scope of some aspects of the present invention. The capacity of the force sensor 20 preferably depends on the expected forces to be experienced by the probe tip 26. In a preferred embodiment of the present invention, for instance, the force sensor 20 is configured to measure forces of up to one thousand, five hundred (1500) N at a resolution of one hundredth (0.01) N.

### Probe

As noted previously, the probe 16 includes the probe tip 26 and the probe shaft 28 to which the probe tip 26 is mounted. More particularly, the probe shaft 28 includes proximal and distal threaded ends 28a and 28b.

The proximal threaded end 28a is received in a corresponding threaded opening (not visible) at the distal end 20b of the force sensor 20 and secured relative thereto by a nut 56.

The probe tip 26 is secured to the distal end 28b of the probe shaft 28. In the illustrated embodiment, for instance, the probe tip 26 is threaded onto the distal end 28b of the probe shaft 28.

Provision of the threaded proximal end 28a of the probe shaft 28 and the associated threaded force sensor opening and nut 56 facilitates adjustment of the length of the probe shaft 28.

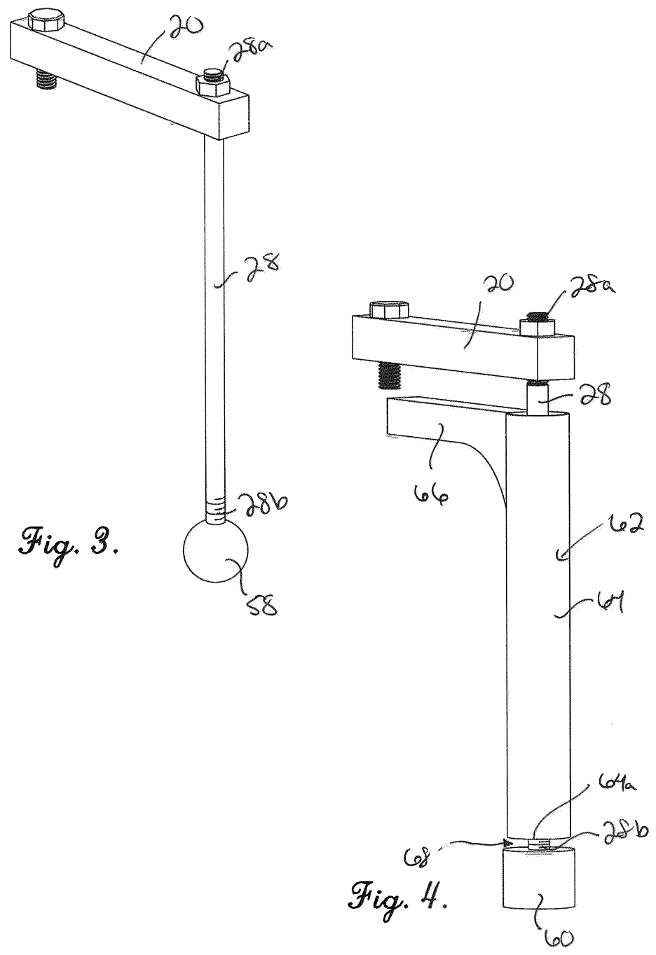

The probe tip 26 in the illustrated embodiment of FIGS. 1 and 2 is in the form of a cone. However, various shapes of probe fall within the scope of some aspects of the present invention. For instance, FIG. 3 illustrates the force sensor 20 and the probe shaft 28 of FIG. 1 with a spherical probe tip 58 mounted to the shaft 28. FIG. 4 illustrates the force sensor 20 and the probe shaft 28 of FIGS. 1-3 with a cylindrical probe tip 60 mounted to the shaft 28.

It is noted that the design of the probe 16 (and, more specifically, of the shaft 28 and the tips 26, 58, and 60) facilitates probe tip interchangeability. For instance, any of the tips 26, 58, and 60 can be readily unscrewed from the threaded distal end 28b of the shaft 28 and replaced by another tip 26, 58, and 60.

Additional tip configurations are also contemplated, including but not limited to semispherical, parabolic, frustoconical, pyramidal, rectangular, bell-shaped, and otherwise curved forms. Conical tips have various semi-angles (e.g., 30°, 45° and 60°) are also permissible with 45° and 60° being preferred.

Probe tip size may also be adjusted as desired.

In general, selection of an appropriate probe tip will be dependent on the shape and size able to provide suitably accurate measurements, as described in greater detail below.

It is also permissible according to some aspects of the present invention for probe tips to be non-interchangeable or for interchangeability to be via an alternative approach. For instance, interlocking components could be provided or other suitably secure yet reversible securement techniques could be used, including but not limited to latches, clips, magnets, and/or adhesives.

Preferably, the probe tips 26, 58, and 60 are manufactured from stainless steel with mirror-grade polish to reduce surface adhesion and improve measurement repeatability. Other materials and finishes fall within the scope of some aspects of the present invention, however.

The probe 16 may in some embodiments include a sleeve 62, as shown in FIG. 4. For instance, the sleeve 62 in the preferred embodiment shown in FIG. 4 includes a cylindrical body 64 that circumscribes and surrounds a substantial portion of the length of the probe shaft 28. The preferred sleeve also includes a sleeve extension 66 that extends parallel to and below the force sensor 20. The sleeve 62 is best suited for use when testing highly flowable mixtures, as the sleeve 62 prevents or substantially restricts material from contacting the probe shaft 28 and potentially confounding test results. Protection against contact with the load cell 20 is also provided.

The sleeve 62 is preferably removable to facilitate use of the probe 16 without the sleeve 62 when not necessary. A permanent sleeve could conceivably be provided, however, or a portion of the illustrated sleeve could be configured for permanent use.

It is noted that, when the sleeve 62 is used, a small gap 68 is provided between a lower end 64a of the sleeve 62 and the associated probe tip (e.g., the probe tip 60, as shown).

### Frame

The frame 12 preferably includes a base assembly 70 and a tower 72. The base assembly 70 preferably includes a base plate 74 and a plurality of legs 76 on which the base plate 74 rests. In a preferred embodiment of the present invention, the legs 76 are adjustable, such that the base assembly 70 is self leveling. Such adjustability is highly advantageous when testing takes place on uneven surfaces.

As shown in FIG. 1, the base plate 74 is configured to receive thereon a sample of the test material 18. In the illustrated embodiment, the sample is provided in a container 78. It is noted, however, that various sample configurations fall within the scope of some aspects of the present invention.

It is also noted that the base plate 74 is preferably removable to facilitate alternative testing modes. On such alternate testing mode will be described in detail below.

The tower 72 provides support and alignment for the penetration system 14. More particularly, the tower 72 includes a foundation plate 80, an upwardly projecting post 82 extending upwardly from the foundation plate 80, and a platform 84 extending laterally from an upper end of the post 82. That is, the platform 84 is supported by the post 82 in a cantilevered manner.

The motor 42 of the actuator 22 is preferably mounted on the platform 84. The actuator shaft 44 is secured to a motor output and extends downward therefrom to be received in an opening 86 in the base plate 74, adjacent to the post 82.

In a preferred embodiment, the actuator shaft 44 extends parallel to the post 82 of the frame 12 and orthogonal to the base plate 74. The probe shaft 28 likewise extends parallel to the threaded actuator shaft 44 and the post 82, and thus orthogonal to the base plate 74. Furthermore, it is most preferred that prior to commencement of material testing, the base assembly 70 is leveled such that the base plate 74 extends horizontally and the post 82, the actuator shaft 44, and the probe shaft 28 each extend vertically. The probe shaft 28 is thereby shiftable into and out of the test material 18 in a controlled vertical orientation (preferably transverse to the surface of the test material 18).

It is noted that frame 12 is preferably sturdy, relatively lightweight, readily portable, and easy to clean, facilitating ease of use in situ.

It is permissible according to some aspects of the present invention for an alternatively configured frame assembly to be provided, however. For instance, the foundation plate could be omitted, the penetration system components could be in some manner suspended rather than supported by a vertical post, and so on. It is most preferred, however, that any such alternative embodiment nevertheless provide sturdy yet portable support.

### Electronics and Computational Components

As noted previously, the electronics and computational components 30 include the controller 32, the force sensor amplifier 34, the motor driver 36, the power supply 38, and the user system 40.

The controller 32 preferably serves as the command center for the device 10. For instance, the controller 32 is preferably configured to control the actuator 22 by regulating operation of the motor 42 based on inputs received from the user via the user system 40. That is, the controller 32 transmits actuation commands to the actuator 22.

As shown in FIG. 2, the motor driver 36 acts as an intermediary between the controller 32 and the motor 42, ensuring control signals are appropriately communicated to the motor 42. More particularly, the motor driver 36 receives low-voltage pulse commands from the controller 32 and delivers precisely timed high-current pulses to the motor 42 to achieve accurate displacement control.

The motor 42 is provided with power from the dedicated power supply 38, ensuring that the required electrical power is available for operation.

The controller 32 is also configured to collect force data from the force sensor 20. More particularly, the controller 32 receives signals from the force sensor amplifier 34, which conditions and amplifies analog signals from the load cell 20 for accurate digital conversion and analysis.

It is preferred that the controller 32 implements a high-frequency sampling protocol (up to 50 Hz, for instance) with adjustable resolution.

Further still, the controller 32 synchronizes data acquisition from the force sensor 20 with motion execution associated with linear motion output of the actuator 22. That is, the controller 32 matches force data resulting from engagement of the probe tip 26 with the material 18 to corresponding displacement data associated with the actuator 22 and the travel of the probe 16. The synchronized force vs. displacement data (i.e. a plurality of synchronized pairs of data) is then transmitted for storage and analysis using the user system 40.

In various examples, the user system 40 includes a user interface 40a and a processor 40b for executing instructions. In some embodiments, executable instructions are stored in a memory device 40c. The processor 40b includes one or more processing units, such as a multi-core processor configuration. The memory device 40c is any device allowing information such as executable instructions and/or written works to be stored and retrieved. The memory device 40c includes one or more computer readable media.

The user system 40 also includes at least one media output component 40d for presenting information to a user, for example, via the user interface 40a. The media output component 40d is any component capable of conveying information to the user. In some embodiments, the media output component 40d includes an output adapter such as a video adapter and/or an audio adapter. An output adapter is operatively coupled to the processor 40b and operatively connectable to an output device such as a display device, for example, and without limitation, a liquid crystal display (LCD), organic light emitting diode (OLED) display, or “electronic ink”display, or an audio output device such as a speaker or headphones.

In some embodiments, the user system 40 includes an input device 40e for receiving input from the user, for example, via the user interface 40a. The input device 40e may include, for example, one or more of a touch sensitive panel, a touch pad, a touch screen, a stylus, a position detector, a keyboard, a pointing device, a mouse, and an audio input device. A single component such as a touch screen may function as both an output device of the media output component 40d and the input device 40e.

Stored in the memory device 40c are, for example, computer readable instructions for providing the user interface 40a to the user via the media output component 40d and, optionally, receiving and processing input from the input device 40e. The computer-readable instructions, when executed by the processor 40b, may cause presentation of controls to configure test parameters such as penetration speed, stroke length, and sampling rate; enable initiation of testing runs; accept optional inputs associated with the material being tested, such as broad type and composition details, environmental conditions under which the test occurs, and a testing location; process data received from the controller 32 to generate outputs such as force-versus-time and/or force-versus-displacement charts and comparative analyses with previous testing sessions; and/or manage archival storage of test configurations, received data, results, and derived analytics.

Via the user interface 40a, the user may configure one or more test parameters (e.g., penetration speed, stroke length, and sampling rate) and initiate testing runs; enter optional information about the material, environment, and location; and view processed results during and after testing, including charts and comparative analyses based on data processed by the processor 40b.

During operation, the processor 40b may provide test-implementation information to the controller 32 (e.g., configured penetration rates and stroke profiles) and process data received therefrom. Outputs generated by the processor 40b may be displayed via the media output component 40d and/or the user interface 40a and may be stored in the memory device 40c. For example, ongoing or recent testing may be shown as force-versus-time and/or force-versus-displacement charts, and comparative analysis with previous testing sessions may be provided.

In a broad sense, the processor 40b is configured to generate, and the user interface 40a is configured to present, information useful in determining (i) what future user inputs to the user interface 40a should be provided (e.g., a recommendation for a different penetration rate, depth, or sampling rate) and/or (ii) what other actions should be considered or taken with respect to the testing process or the material 18 (e.g., recommendations for modification of material content such as increasing water content or decreasing aggregate; notifications of unfavorable environmental conditions such as detrimental heat, cold, or humidity; or a suggestion to use an alternative probe tip). These determinations and recommendations are based on data received from the controller 32 and processed by the processor 40b, and may be archived in the memory device 40c for later review and comparison.

The user system 40 may also include a communication module 40f, which is communicatively connectable to a remote device such as the controller 32 via wires, such as electrical cables or fiber optic cables, or wirelessly, such as radio frequency (RF) communication. The communication module 40f may include, for example, a wired or wireless network adapter or a wireless data transceiver for use with Bluetooth communication, RF communication, near field communication (NFC), and/or with a mobile phone network, Global System for Mobile communications (GSM), 5G, or other mobile data network, and/or Worldwide Interoperability for Microwave Access (WiMax) and the like.

A preferred mathematical approach associated with such data analysis will be discussed in greater detail below.

It will be readily apparent to those having ordinary skill in the art that various steps and components described above may require use of one or more additional processors or other electronics or computational components (not shown) beyond those illustrated and described. Integration of components is also permissible. That is, FIG. 2 and the description above provide a general description of the computational and electronics elements that facilitate the inventive method but should be understood to be broad and non-limiting in nature.

It is also noted that connections between the various components, including the user system 40, the controller 32, the motor driver 36, and the force sensor amplifier 34, are preferably bi-directional, enabling real-time feedback and execution.

### Method of Use

Broadly characterized, in a preferred method of use, the probe tip 26 is inserted into the material 18 to be characterized, and force vs. displacement readings are taken.

More particularly, a sample of the material 18 is provided in the container 78 that is positioned on the base plate 74 of the frame 12. If necessary, the legs 76 are adjusted to ensure the base plate 74 is level.

A user provides inputs to the user system 40, as described previously. Necessary ones of these inputs (e.g., desired penetration speed and depth) are provided to the controller 32, which in turn transmits instructions to the motor driver 36.

Such necessary inputs could, in some methods, include directly applicable inputs such as desired penetration speed and depth. In other methods, a user could instead provide relevant, indirectly useable inputs, such as inputs regarding the material type, ambient conditions, time since pour, etc. The processor 40b could then automatically determine the penetration speed and depth for testing and provide corresponding information to the controller 32.

The motor driver 36 controls the motor 42, which in turn rotates the actuator shaft 44. Rotation of the actuator shaft 44 results in upward or downward shifting of the adapter 24 and, in turn, the probe 16, at a speed corresponding to the rotational velocity of the actuator shaft 44.

When the probe tip 26 contacts the surface of the material 18, the material 18 exerts a force on the probe tip 26. Continued insertion of the probe 16 results in further submersion of the probe tip 26 and continued application of forces thereonto by the material 18.

In one preferred method of testing, commencement of force recordation begins immediately upon contact of the probe tip 26 with the surface of the material 18. That is, the force sensor 20 begins to output signals corresponding to the sensed forces (and/or recordation of the output signals and corresponding forces begins) immediately upon engagement of the tip 26 with the material 18. Full submersion of the probe tip 26 may or may not eventually occur, although full submersion is preferred. This method is particularly suitable when the thickness of the material being tested is small, such as in 3D-printed layers.

It is noted that “submersion” as used herein does not necessarily require a filling-in of material 18 above the probe tip 26. However, the entirety of the probe tip 26 should be disposed below the initial material surface.

In another preferred method of testing, commencement of force recordation begins only after the tip 26 is fully submerged, as described above. That is, although the tip 26 is subject to forces immediately upon contact with the material 18, it is only after complete submersion of the tip 26 has first occurred that the force sensor 20 begins to output signals corresponding to the sensed forces and/or that the output signals and corresponding forces are recorded. (Of course, continued lowering of the now submerged tip 26 to even greater depths leads to continued subjection to forces and corresponding transmittal of associated signals.)

It is noted that, in tests featuring full submersion, it is preferred that the probe 16 stop briefly when full submersion has occurred. The force-acquisition portion of the test begins thereafter.

Received force signals are amplified by the force signal amplifier 34 and transmitted to the controller 32. The controller 32 pairs the received force data with corresponding penetration depth data points to form synchronized pairs, which are then transmitted by the controller 32 to the processor 40b associated with the user system 40.

It is noted that penetration depth data may be acquired by several different methods, including but not limited to methods combining force sensor 20 deflection data, actual actuator motion data, and/or instructional data pertaining to depth sent from the controller to the actuator.

When the desired penetration depth is achieved, the actuator motion is reversed via a signal from the motor driver 36 until the probe 16 returns to its initial position.

It is noted that, in a preferred method of testing, penetration to the desired maximum depth takes less than about one hundred twenty (120) seconds, more preferably less than about sixty (60) seconds, and most preferably less than about thirty (30) seconds. Penetration times at the upper end of these preferred ranges are more preferably associated with full submersion of the tip 26, whereas those at the lower end of these preferred ranges are more preferably associated with penetration of the material 18 by the tip 26 without eventual complete submersion.

In a preferred method of testing, commencement of penetration begins any time after mixing but before the setting time of the material, which varies depending on the composition of the material and certain environmental factors such as temperature and humidity. For some material compositions, for instance, commencement of testing preferably begins less than about three (3) hours, more preferably less than about two (2) hours, and most preferably between about fifteen (15) minutes and about ninety (90) minutes after preparation of the material. Again, however, the preferred testing window will vary according to the specific material composition and other factors.

It is noted that, as used herein, “preparation of the material” refers to presentation of the fully mixed material in its desired testing location. In the embodiment of FIGS. 1 and 2, for instance, material preparation is complete when the container 78 has received the sample of the material 18. For testing directly in a drum, preparation would be deemed complete simply upon sufficient mixing of the various components in the drum.

Force vs. displacement data, recommended testing modifications, recommended material composition changes, and/or other information is preferably presented to the user via the user interface 40a as it comes available. For instance, real-time force vs. displacement data is preferably provided, whereas recommendations are made only after data acquisition is complete.

In one preferred method, adjustments to testing parameters, material composition, and so on are only made based on direct input from a user. Adjustments may be made to one or more valves, augers, paddles, hoppers, drums, and more, for instance, that collectively constitute a material mixing or preparation system. An example of such a system and how adjustments can be made thereto to change the produced material will be described in greater detail below.

In another preferred method, various adjustments may be made automatically, without direct or ongoing user input. For instance, testing data and/or other inputs could be used to generate automated adjustments to probe speeds or testing depths to facilitate more accurate testing. Such data could alternatively be used to send signals (e.g. from the controller) to the material mixing system to adjust the proportions of the material—e.g., through decreasing liquid input or increasing aggregate input. That is, it is permissible according to some aspects of the present invention for the penetration device and a material mixing or preparation system to be integrated. Again, an example of such a system and how adjustments can be made thereto to change the produced material will be described in greater detail below.

### Second Embodiment: Rotationally Actuated Penetration Device Mounted to Print Head

A second preferred embodiment of the present invention, in which a pair of penetration devices 110 and 112 are mounted on a three-dimensional concrete printer 114 (alternatively referred to as a 3D concrete printer or EDCP), is illustrated in FIG. 5.

It is initially noted that, with certain exceptions to be discussed in detail below, many of the elements of the penetration devices 110 and 112 are the same as or very similar to those described in detail above in relation to the penetration device 10 of the first preferred embodiment. Therefore, for the sake of brevity and clarity, redundant descriptions and numbering will be generally avoided here. Unless otherwise specified, the detailed descriptions of the elements presented above with respect to the first embodiment should therefore be understood to apply at least generally to the second embodiment, as well.

Among other things, the printer 114 broadly includes a print head 116. The print head 116 includes an extruder 118 and a nozzle 120 through which a printable material 122 is expressed to form a printed filament 124.

The penetration device 110 preferably includes a frame 126 and a penetration system 128. The penetration system 128 includes a probe 130, a force sensor 132, an actuator 134, and an adapter 136. The frame 126 includes a tower 138, a foundation plate 140, and a platform 142. The probe 130 includes a probe shaft 144 and probe tip 146. The actuator 134 includes a motor 148, a coupling 150, and an actuator shaft 152.

Similarly, the penetration device 112 preferably includes a frame 154 and a penetration system 156. The penetration system 156 includes a probe 158, a force sensor 160, an actuator 162, and an adapter 164. The frame 154 includes a tower 166, a foundation plate 168, and a platform 170. The probe 158 includes a probe shaft 172 and a probe tip 174. The actuator 162 includes a motor 176, a coupling 178, and an actuator shaft 180.

As will be apparent from the above, the penetration devices 110 and 112 are similarly configured to the penetration device 10 except through omission of the base plate and legs of the frame and through removal of the individual controllers. More particularly, with regard to the latter, the controllers of the penetration devices 110 and 112 are instead preferably integrated into a unified controller (not shown) that also provides signals to the concrete printer 114.

As will be readily understood by those having ordinary skill in the art, 3D concrete printing using the printer 114 is an additive manufacturing process in which a specialized concrete 122 is extruded by the extruder 118 and output via the nozzle 120 to form a printed filament 124. A given layer 124a of the printed filament 124 is then overlaid by another layer 124b of extruded concrete 122 (that is, a subsequent layer 124b of concrete 122 is added on top of a prior layer 124a of concrete 122 so as to be stacked on top of the first layer 124a), with the process repeating until a desired dimension is achieved. Thus, the printer 114 forms a structure 182 comprising a plurality of vertically stacked concrete layers (such as layers 124a, 124b) that cure over time. In the illustrated embodiment, for instance, the printed filament 124 forms a plurality of layers 124a-124g, with layer 124g being in progress.

It is noted that printing discontinuities may occur to facilitate easy placement of doors, windows, etc. Exterior walls, interior walls, and/or other features may be printed in this manner, in accordance with the overall home or building design plan.

The position of the print head 116 is preferably automated based on a pre-programmed path corresponding to the design of the home or other structure 182, as created in or provided in a 3D modeling program. Start and stop of printing in association with desired structural discontinuities is also typically pre-programmed in keeping the structural design. The unified controller (not shown) provides necessary signals to implement the designed program.

The concrete 122 preferably comprises a combination of dry matter and liquid, which are combined in an upstream hopper or mixer (not shown). For instance, in one conventional configuration, the dry matter (including cement and aggregates, for instance) is dispensed into the mixer. Water is added into the mixer, which mixes the water and dry matter together to form the concrete. Additives such as plasticizer, fibers, and more may also be included to alter the final or temporary properties of the concrete, including but not limited to the concrete's strength, workability, water requirements, set time, and longevity. The concrete is then pumped to the print head 116.

Various alternatives to the above general configuration are permissible without departing from some aspects of the present invention. For instance, mixing could occur in a mobile rotating drum rather than fixed hopper, or the extruder could be preceded by a small tank from which concrete is fed to the print nozzle via a screw feeder. Various valves, augers, paddles, hoppers, drums, and more may also form part of the overall system.

As noted above, manual (e.g., via a user) or automated (e.g., based on signals transmitted by the controller 32) adjustments to the concrete composition can be made based on results of testing. For instance, a valve associated with a water or liquid source (e.g., disposed in a pipe running from such source to a mixer) could be opened, closed, or adjusted to modify the liquid proportion of the concrete mix. A conveyor or auger associated with any one or more of the dry matter components could be stopped, started, sped up, or slowed to modify the dry matter contents. Valves associated with any one or more of the dry matter components could be opened, closed, or otherwise; and/or a speed or direction of a mixer motor could modified. Heating or cooling elements could be turned on or off or adjusted, as could ventilation devices. That is, any mechanical or structural component associated with the material preparation process may, in some embodiments, be manually or automatically controlled based on testing results.

Initial states of the various mechanical or structural components may also be similarly controlled or determined.

As will be apparent to those of ordinary skill in the art, proper composition of the mixed concrete 122 is essential to a successful printing operation. If the mixed concrete 122 is highly flowable (either due to added water or chemical admixtures, for instance), pumpability and flow through the print head 116 will be excellent; but the printed material or filament 124 may be too soft and flowable to retain its shape, cure in a timely manner, and provide required structural properties. In contrast, if the mixed concrete 122 is too stiff or thick (e.g., having too low a relative water content), pumpability and flow through the print head 116 will be impossible, difficult, slow, inconsistent, discontinuous, or otherwise unsuitable for a controlled print. A poor concrete mix may also lead to undesirable aesthetic effects in the printed structure 182.

It is also noted that changing conditions may result in what is initially an ideal or suitable composition gradually or rapidly becoming ill-suited to the application. For instance, environmental changes might dictate that modification of the water content of the printed filament 124 is desirable during the course of a multi-hour print, or provision of additives to accelerate curing may be indicated. In some cases, a change in print speed may also be beneficial, allowing for lower layers to more fully cure before additional layers are added, for instance.

Although those skilled in the art may make necessary or preferred adjustments to the concrete mixture based on experience and/or slump testing, such adjustments lack precision and scientific rigor. Rigorous analysis of a concrete specimen may be made using a conventional rotational (vane) rheometer, but such analysis is time consuming and requires a non-portable lab setup.

In contrast, the present penetration devices 110 and 112 and method are well suited for flexible, rapid, on-site testing and quality control of concrete 122 associated with 3D concrete printing applications. More particularly, the first one of the penetration devices 110 is disposed on a fore side of the print head 116 (i.e., ahead of the print head 116 direction of motion) so as to lead the print head 116. The second one of the penetration devices 112 is disposed opposite the first penetration device 110. That is, the second penetration device 112 is disposed on an aft side of the print head 116 (i.e., behind the print head 116 direction of motion) so as to follow the print head 116.

In greater detail, the printer 114 preferably includes a mounting bracket 184 to which the penetration devices 110 and 112 are mounted to the printer 114. More particularly, in an illustrated embodiment, the mounting bracket 184 includes a generally horizontally extending positioning structure 186 and a pair of generally vertically extending mounting plates 188 and 190 extending orthogonally from the positioning structure 186. The penetration device 110 is preferably secured to the mounting plate 188, whereas the penetration device 112 is preferably secured to the mounting plate 190.

In another preferred embodiment of the present invention, the mounting bracket is configured to rotate independently around the vertical axis, thereby enabling each penetration device to cover a broader range of testing locations.

In such a configuration, a single mounting plate could also be sufficient, as the rotational capability of the bracket allows the device to be aligned with successive printed layers, permitting tests to be conducted sequentially.

Securement may be by any of a variety of means, including but not limited to discrete fasteners, latches, hook-and-loop, adhesives or glues, magnets, interlocking components, slidable track, etc.

Other approaches to mounting of the penetration device on or near the print head fall within the scope of some aspects of the invention as well, however.

As will be readily apparent from the above, the embodiment of FIG. 5 enables on-site testing and monitoring of the rheological properties of the concrete 122 at various locations and stages during printing. For instance, the lead penetration device 110 is well suited for penetrating and testing the concrete 122 immediately ahead of the print head 116. That is, the penetration device 110 is well suited for penetrating and testing a previously printed layer (e.g., the layer 124f in the illustrated embodiment). In contrast, the aft or rear penetration device 112 is well suited for testing the concrete 122 immediately behind the print head 116. The penetration device 112 is consequently well suited for testing a just printed portion of a current or top layer (e.g., the layer 124g in the illustrated embodiment).

Data acquired from these tests can be analyzed as broadly described above and as will be described in greater mathematical detail below, then used to make on-site adjustments to the concrete composition to achieve improved structural results.

It is noted that data acquired from testing of both freshly printed filament and previously printed filament can be extrapolated to estimate the properties of even earlier printed filament and the continually changing properties of the sampled filament, providing valuable information on the properties of the structure as a whole over time.

Although FIG. 5 illustrates a preferred printer/probe configuration, it is noted that alternative configurations of printer/probe combinations fall within the scope of some aspects of the present invention. For instance, it is permissible for a penetration device to be located only on an aft side of a print head. This positioning facilitates testing of just-printed concrete and permits real-time data collection and analysis of actual building material. Adjustments may then be made as necessary.

Alternatively, it is permissible for a penetration device to be mounted only on a fore side of a print head, to enable testing of previously printed concrete from the preceding (i.e., lower) layer. Such testing enables monitoring of the curing process of the actual building material.

In yet another alternative embodiment of the present invention, a penetration device is used to test concrete in a hopper just prior to pumping of the concrete to the extruder and print head. Alternatively, a sample may be removed from the hopper or elsewhere and tested separately. Testing at a pre-print stage in this manner permits adjustments to the composition to be made upstream of the pump and therefore prior to pumping and printing of the material. Clogs and other potential problems may in this manner be typically avoided.

Various other testing locations and techniques are also permissible, including but not limited to laterally oriented testing of any desired print layer, vertically oriented testing through multiple layers, and so on.

In general, however, each of the above-described use cases provides real-time monitoring and analysis of the concrete properties, enabling an operator or an automated system to adjust various material and/or process parameters in response.

### Third Embodiment: Linearly Actuated Probe Device

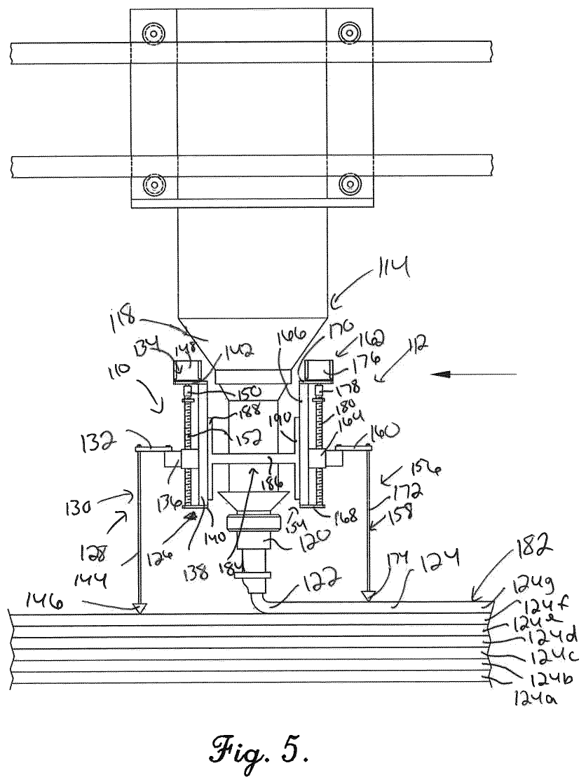

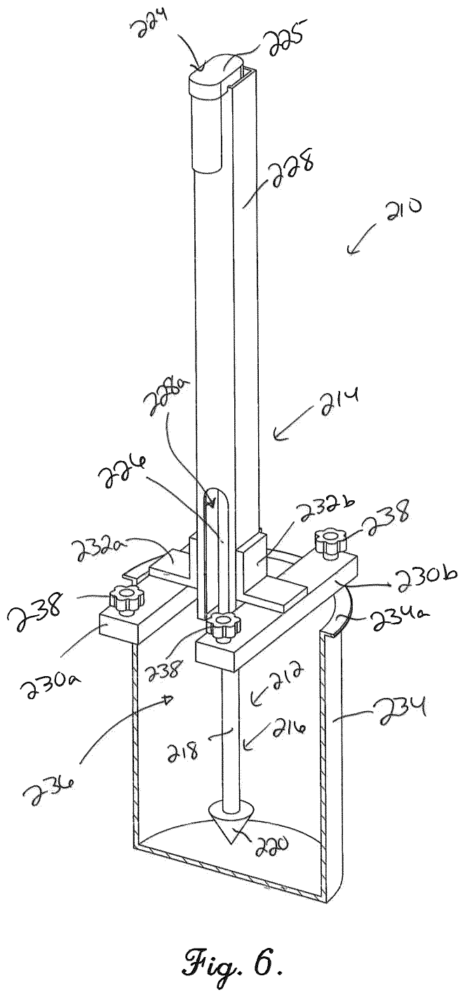

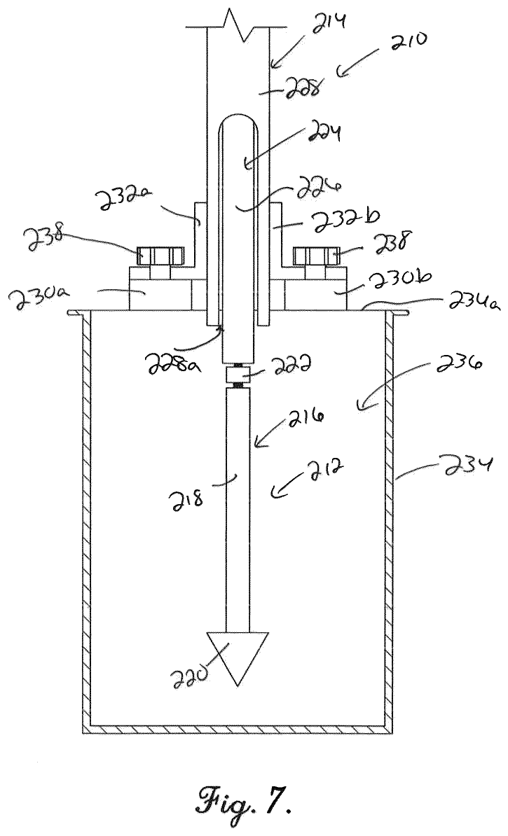

A third preferred penetration device is illustrated in FIGS. 6 and 7. It is initially noted that, with certain exceptions to be discussed in detail below, many of the elements of the penetration device 210 are the same as or very similar to those described in detail above in relation to the penetration device 10 of the first preferred embodiment and the penetration devices 110 and 112 of the second preferred embodiment. Therefore, for the sake of brevity and clarity, redundant descriptions and numbering will be generally avoided here. Unless otherwise specified, the detailed descriptions of the elements presented above with respect to the first and second embodiments should therefore be understood to apply at least generally to the third embodiment, as well.

Turning now to FIGS. 6 and 7, the penetration device 210 includes a penetration system 212 and a housing or frame 214. The penetration system 212 includes a probe 216 comprising a probe shaft 218 and a probe tip 220. The penetration system 212 also includes a force sensor 222 and an actuator 224.

In contrast to the rotary actuators 22, 134, and 162 of the first and second embodiments, the actuator 224 is a linear actuator. More particularly, the actuator 224 includes an integrated motion generator 225 and motor driver (not shown), an integrated motor controller (not shown), and an output structure 226.

In a preferred embodiment, the motion generator 225 is a DC motor or stepper motor, with the motor driver regulating motor speed and direction according to commands from the controller. For instance, in a preferred method of regulation, the motor driver uses pulse-width modulation (PWM) for speed control and encoder feedback for position tracking, if required.

The output structure 226 is preferably a non-threaded shaft, as shown.

An external power supply (not shown; preferably a DC supply) may provides power to the motor controller, although other sources of power are within the ambit of this embodiment.

The force sensor 222 is provided linearly between (i.e., axially between) the output structure 226 of the actuator 224 and the probe shaft 218 and is coupled to each such that linear translation of the actuator output shaft 226 is transferred to the probe 216.

Preferably, the stroke length of the actuator is variable. Most preferably, the stroke length is up to five hundred (500) mm. Such variability accommodates testing at a broad range of different depths.

The force sensor 222 preferably senses forces applied to the probe tip 220 in a manner generally similar to that described above. However, the force sensor 222 in the illustrated embodiment of FIGS. 6 and 7 is a pancake-type load cell, rather than a bending beam load cell.

Load cell data are preferably collected via a dedicated acquisition module (not shown) for processing and analysis.

The frame or housing 214 preferably includes an at least substantially vertical tube 228; a pair of parallel beams 230a and 230b extending at least substantially orthogonally relative to the tube 228; and a pair of brackets 232a and 232b connecting respective ones of the beams 230a and 230b to the tube 228.

The tube 228 is preferably a hollow tube 228 defining a lumen 228a through which the actuator shaft 226 extends. The tube 228 in the illustrated embodiment is generally rectangular in cross section, although other shapes (e.g., circular in cross section) are permissible.

In a preferred embodiment, the probe shaft 218 comprises stainless steel and the frame tube 228 comprises aluminum. However, other materials fall within the scope of some aspects the present invention.

Furthermore, although the illustrated brackets 232a and 233b are L-shaped brackets, alternative bracket shapes or interconnection means for securing the beams to the tube fall within the scope of some aspects of the present invention. Among other things, for instance, the beams and tube could be bolted, latched, adhered, welded, or otherwise secured to each other.

The penetration device 210 as shown in FIGS. 6 and 7 is configured for mounting on a container or bucket 234 and subsequent testing of a sample of a material (not shown) contained in an internal space 236 defined by the bucket 234. It is noted, however, that various sample configurations fall within the scope of some aspects of the present invention. For instance, the sample could be provided amorphously on a plate; contained in an alternative container; or as described in greater detail below, part of an in-progress project (e.g., a just-poured foundation slab).

In contrast to the specialized printable concrete 122 of the second embodiment, the material of the third preferred embodiment is preferably a conventional concrete, such as a self-consolidated concrete (SCC) or conventionally vibrated concrete commonly used in conventional construction processes (e.g., foundation or formwork pouring, etc.). Testing of other materials, including but not limited to printable concretes, is permissible, however.

In the illustrated embodiment, the beams 230a and 230b extend laterally across the container 234 and above the chamber 236, such that the probe 216 is disposed above or within the chamber 236 (and thus positionable above or within any material received in the chamber 236).

In greater detail still, the container 234 preferably includes an upper edge 234a. The beams 230a and 230b rest on the upper edge 234a. A plurality of lock screws 238 are provided to secure the beams 230a and 230b against the upper edge 234a.

It is noted, however, that alternative container configurations fall within the scope of some aspects of the present invention. Furthermore, alternative locking mechanisms could be provided, including but not limited to bolts or other discrete fasteners, latches, interlocking elements, and adhesives. Locking mechanisms could also be omitted entirely.

Testing of the material 236 follows a procedure similar to that described above with regard to the first penetration device 10.

### Fourth Embodiment: Big Probe Mounted Over Slab

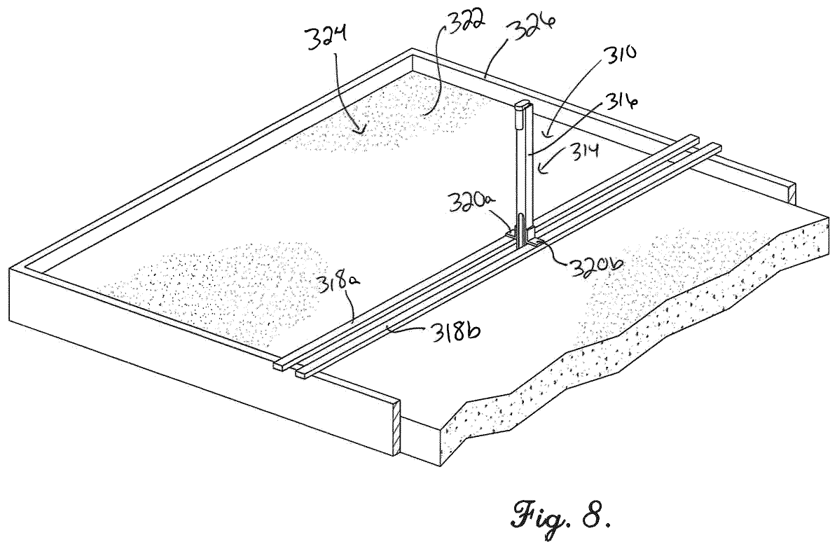

A fourth preferred penetration device is illustrated in FIG. 8. It is initially noted that, with certain exceptions to be discussed in detail below, many of the elements of the penetration device 310 of the fourth embodiment are the same as or very similar to those described in detail above in relation to the devices 10, 110, and 210 of the first through third embodiments, respectively. Therefore, for the sake of brevity and clarity, redundant descriptions and numbering will be generally avoided here. Unless otherwise specified, the detailed descriptions of the elements presented above with respect to the first through third embodiments should therefore be understood to apply at least generally to the fourth embodiment, as well.

Similarly to the penetration device 210, for instance, the penetration device 310 also includes a frame 314. More particularly, the frame 314 preferably includes a tube or tower 316, a pair of beams 318a and 318b, and a pair of brackets 320a and 320b. However, whereas the beams 230a and 230b of the frame 214 of the third preferred embodiment are relatively short, the beams 318a and 318b of the frame 314 are significantly elongated. This alternative frame structure enables setup of the penetration device 310 directly within a jobsite for in situ testing of an actual construction material 322 currently forming a structure, rather than simply a sample of material intended for use in forming a structure.

In the illustrated embodiment, for instance, a slab 324 of material 322 has been poured into a form or framing 326. The beams 318a and 318b extend across the slab 324 to rest on the framing 326, with the penetration system 314 being suspended above an intermediate or central portion of the slab 324 to facilitate direct testing of a “typical” portion of the material 322 thereof.

It is noted that, although the illustrated beams 318a and 318b are of fixed length and orientation, it is permissible according to some aspects of the present invention for the beams to be length-adjustable, pivotable, and or otherwise adjustable to accommodate testing in various on-site configurations.

Furthermore, it falls within the scope of some aspects of the present invention for the remaining components of the penetration device to be repositionable along the beams. For instance, the tower and the brackets could be slidable along the beams, or the beams could include a plurality of discrete attachment locations for the brackets and tower. Such adjustability could provide highly beneficial consistency-testing capability, allowing the material to be tested at locations throughout the broader slab or area.

### Theoretical Framework for Calculating the Yield Stress of a Tested Material

In any of the preferred embodiments and methods of the present invention, an associated processor, such as the processor 40b, is preferably configured to analyze data received from an associated controller, such as the controller 32, to provide useful output to a user system, such as the system 40, or to guide automated adjustments to testing parameters, material composition, etc. As will be readily apparent to those having ordinary skill in the art, such data includes but is not limited to force vs. displacement data.

In a preferred methodology, such processor applies a novel theoretical framework to calculate the static yield stress of the tested material. In greater detail, a solid plasticity approach to calculating the static yield stress is preferably used (e.g., as opposed to a force equilibrium or fluid dynamic approach, for instance.) More particularly, penetration of the probe into the material is modeled as a bearing capacity problem.

In this method, the penetration force is assumed to be equal to the collapse load of a conical foundation on soil. The collapse load (or bearing capacity) of a foundation is determined using a limit equilibrium method in which it is assumed that the foundation force induces a state of plastic equilibrium in the material, resulting in the formation of an unstable mechanism where part of the material slips relative to the rest of the mass.

Upon application of this framework and simplification where feasible, the penetration force of the conical penetration tip 26 of radius r at a depth of h into the material sample 18 may be expressed as a function of static yield stress of the material, geometry of the cone, and bearing capacity factor for cohesion (Nc).

In a full penetration test (i.e., with full submersion of the tip 26), the yield force is determined from a force vs. displacement curve. Then the yield force is divided by the base area of the tip 26 (e.g., of the cone forming the tip 26) as well as the Nc of the material to calculate the static yield stress of the material.

In a tip penetration test (i.e., without full submersion of the tip 26), a stress vs. displacement curve is generated instead of a force vs. displacement curve. This is achieved by dividing the penetration force by the cross-sectional area of the tip 26 (e.g., of the cone forming the tip 26) at the corresponding depth (Apen). The stress initially begins at a high value due to the extremely small Apen at shallow depths. However, as the penetration depth increases and Apen becomes sufficiently large, the stress approaches an equilibrium value, which remains constant until the probe is fully submerged. In the tip penetration test setup, the determination of static yield stress is based on the equilibrium stress observed during the tip's submergence. The equilibrium stress is divided by the Nc for the material to calculate the static yield stress.

The selection of the correct bearing capacity factor NC requires knowledge of the angle of internal friction of the material being tested. The angle of internal friction can be used to look up an appropriate cohesion bearing capacity factor through reference to well-known soil mechanics data tables, and the yield stress may then be calculated.

In some instances, the angle of internal friction can be estimated or neglected. Because 3D print materials are rich in paste, for instance, one approach is to treat such materials as plastic materials whose behavior is dominated by cohesion rather than frictional contact between particles. In such circumstances, the angle of internal friction may be neglected without introducing substantial bias.

### Summary of Testable Materials, Testing Modalities, and Data Applications

As will be readily understood by those having ordinary skill in the art, freshly poured concrete is known as “plastic concrete” or “fresh concrete.” In this initial stage, the material is workable and wet and can be poured and shaped.

The concrete then begins to gradually lose its fluidity and to stiffen in a process known as setting. More particularly, ASTM C125-20: Standard Terminology Relating to Concrete and Concrete Aggregates, describes the concrete setting process as “the process, due to chemical reactions, occurring after the addition of mixing water, that results in a gradual development of rigidity of a cementitious mixture.” Setting typically takes between four (4) and twenty-four (24) hours, although the time range is variable depending on factors including but not limited to the concrete mixture and the environmental parameters. During this stage, the concrete can be referred to as “setting concrete”; and when setting is complete, the concrete is generally hard or rigid to the touch but does not have its final strength.

During the next stage, known as curing, the concrete is maintained at appropriate moisture and temperature levels to allows it to gain strength and durability over time. The material eventually becomes a durable, rock-like material referred to as “hardened concrete.”

The various embodiments and methods of the present invention described above are well suited for testing and evaluation of fresh or partially set cementitious materials in laboratory, industrial, and field conditions. Both printable and conventional materials are suitable. For instance, the present invention is well suited for testing a variety of cementitious composites, including near-zero or zero-slump concrete, 3D-printable concrete, conventional concrete, and self-consolidating concrete (SCC), with or without fiber reinforcement.

Additional materials may also be tested and analyzed without departing from the scope of some aspects of the invention. For instance, a penetration probe in accordance with the present invention could be used to test printable mortars, clays, bio-based materials (comprising wood fibers, plant-based resins, sawdust, and/or other natural elements), polymer composites, recycled plastics, industrial or agricultural waste products, etc.

As will be readily apparent to those of ordinary skill in the art, results from testing of such alternative materials may require modified post-testing mathematical analysis compared to that associated with the above-described tests. Furthermore, in some instances, changes to the probe size or other parameters may be warranted. However, the broad inventive concepts described herein will nevertheless be applicable.

Various testing modalities are supported by the present invention, including but not limited to in-situ testing directly on slabs, pavements, or freshly cast structural elements; in-container testing using molds, buckets, etc. ; and in-line monitoring when mounted on a 3D concrete printer.

Rheological analysis using testing data combined with the above-referenced theoretical framework can be used to determine rheological properties of a given material; and resulting data can be used for a variety of analyses. These include but are not limited to verification of workability, consistency, and batch uniformity on site or at batching plants; assessment of bleeding in highly flowable concrete; detection of segregation in SCC and other flowable concretes through depth profiling; bond window determination for multi-lift placements in layered construction; and mix design optimization and performance verification of chemical and mineral admixtures in a laboratory.

### Conclusion

Features of one or more embodiments described above may be used in various combinations with each other and/or may be used independently of one another. For instance, although a single disclosed embodiment may include a preferred combination of features, it is within the scope of certain aspects of the present invention for the embodiment to include only one (1) or less than all of the disclosed features, unless the specification expressly states otherwise or as might be understood by one of ordinary skill in the art. Therefore, embodiments of the present invention are not necessarily limited to the combination(s) of features described above.

The preferred forms of the invention described above are to be used as illustration only and should not be utilized in a limiting sense in interpreting the scope of the present invention. Obvious modifications to the exemplary embodiments, as hereinabove set forth, could be readily made by those skilled in the art without departing from the spirit of the present invention.

Although the above description presents features of preferred embodiments of the present invention, other preferred embodiments may also be created in keeping with the principles of the invention. Furthermore, as noted previously, these other preferred embodiments may in some instances be realized through a combination of features compatible for use together despite having been presented independently as part of separate embodiments in the above description.

The inventors hereby state their intent to rely on the Doctrine of Equivalents to determine and access the reasonably fair scope of the present invention as pertains to any apparatus not materially departing from but outside the literal scope of the invention set forth in the following claims.

1. A method of acquiring and analyzing properties of a fresh or partially set cement-based material, said method including the steps of:
(a) penetrating the material with a probe driven at a constant penetration speed by an actuator regulated by a controller;
(b) for each of a plurality of sampling times, measuring, with a force sensor, a penetration force applied to the probe;
(c) transmitting, from the force sensor, force data associated with the measured penetration forces to the controller;
(d) for each of the plurality of sampling times, determining, by the controller, a probe penetration depth based on one or more of the following: the force data and actuator position data, wherein the actuator position data is received from the actuator or inferred by the controller;
(e) recording, by the controller, the determined probe penetration depth;
(f) for each of the plurality of sampling times, associating, by the controller, the probe penetration depth and the penetration force to form a synchronized pair;
(g) determining, by a processor, a yield force of the material based on change in relationship between the penetration depth and the penetration force within the synchronized pairs; and
(h) calculating a static yield stress of the material by the processor, based at least in part on the yield force, a probe geometry factor, and an angle of internal friction of the material.

2. The method of claim 1, further including the step of:
(i) displaying test data and analysis by a user interface.

3. The method of claim 1, further including the step of:
(j) adjusting a material composition parameter based at least in part on the static yield stress calculated by the processor in step (h).

4. The method of claim 3,
step (j) including the step of adjusting one or more valves, drums, augers, paddles, or hoppers to facilitate adjustment of said material composition parameter.

5. The method of claim 4,
said controller initiating such adjustments.

6. The method of claim 1, further including the step of:
(k) presenting testing adjustments by a user interface, based at least in part on analysis by the processor of the static yield stress calculated in step (h).

7. The method of claim 1,
said probe including a probe tip,
step (a) including the step of fully penetrating the material with the probe tip such that the probe tip is immersed in the material.

8. The method of claim 1,
step (a) commencing between immediately after preparation of said material and prior to a setting time of the material.

9. The method of claim 1,
step (h) including the steps of fitting a curve to the penetration depths and the corresponding penetration forces by the processor, identifying a final at least substantially linear region of the curve and fitting a line thereto by the processor, and identifying an inflection point at which the curve deviates from the line by the processor.

10. The method of claim 1,
said constant penetration speed being between about 0.5 mm/s and about 15 mm/s.

11. The method of claim 1,
step (a) having a duration of less than about 60 seconds.

12. The method of claim 1, further comprising the steps of:
(l) prior to commencement of step (a), mounting said probe and a second probe to a printhead of a concrete printer; and
(m) after completion of step (1), implementing each of steps (a)-(h) using said probe and, at least substantially contemporaneously, implementing each of steps (a)-(h) using said second probe.

13. The method of claim 12,
said printhead printing a plurality of vertically stacked layers of said material,
step (m) including the step of said probe and said second probe testing respective ones of said layers.

14. A portable penetration device for in-situ testing and characterization of a cement-based material, said penetration device comprising:
a probe configured to penetrate the material;
an actuator for controlling vertical displacement of the probe;
a force sensor mounted relative to the probe and configured to measure forces exerted on the probe;
a controller for synchronizing force and displacement data associated with the probe to form synchronized pairs; and
a processor for calculating and outputting a yield stress based on the synchronized pairs.

15. The device of claim 14,
said actuator including an actuator shaft and an adapter mounted to and linearly shiftable along the actuator shaft,
said force sensor comprising a bending beam load cell,
said load cell presenting first and second load cell ends,
said probe extending from a first one of said load cell ends,
said adapter supporting a second one of said load cell ends.

16. The device of claim 14,
said probe including a probe shaft,
said actuator including an actuator shaft aligned axially with the probe shaft,
said force sensor being disposed axially between the probe shaft and the actuator shaft.

17. The device of claim 14,
said probe including—
a probe shaft, and
a probe sleeve configured to restrict flow of the material onto the probe shaft.

18. The device of claim 14, further comprising:
a self-leveling frame including a base plate and a plurality of adjustable legs supporting the base plate.

19. The device of claim 14, further comprising
a frame including a beam extending at least substantially orthogonally relative to the probe,
said beam configured to position the probe relative to the material.

20. The device of claim 14, further comprising:
a frame including a post extending at least substantially parallel to the probe,
said post facilitating mounting of the portable penetration device to a print head of a three-dimensional concrete printer.
